(19) United States
(12) Patent Application Publication
(10) Pub. No.: US 2011/0295138 A1
(43) Pub. Date: Dec. 1, 2011

(54) Method and system for reliable inspiration-to-expiration ratio extraction from acoustic physiological signal

(76) Inventors: Yungkai Kyle Lai, Aliso Viejo, CA (US); Yongji Fu, Vancouver, WA (US)

(21) Appl. No.: 12/800,932

(22) Filed: May 26, 2010

(51) Int. Cl. A61B 5/08

(52) U.S. Cl. 600/529

(57) ABSTRACT

A method and system for reliably estimating inspiration-to-expiration ratio from an acoustic physiological signal. A background sound level is set to an energy level whereat a predetermined share of data points on an energy envelope is below the energy level, after which respiration phase start and end times are determined at energy crossings above the background sound level, enabling more reliable determination of respiration phases. Moreover, reliably determined respiration phase start and end times, in addition to being used to estimate inspiration-to-expiration ratio, are applied to other purposes, such as estimating respiration period, validating an independently computed respiration period and/or adjusting a sampling window of the acoustic physiological signal, reducing system complexity and conserving computational resources.

### BACKGROUND OF THE INVENTION

The present invention relates to respiration parameter extraction and, more particularly, to a method and system for reliably extracting inspiration-to-expiration ratio from an acoustic physiological signal.

Real-time monitoring of the physiological state of human subjects is in widespread use in managing cardiovascular, pulmonary and respiratory disease, and is also widely used in other contexts such as elder care. Some real-time physiological monitoring devices monitor physiological state by capturing and evaluating acoustic signals containing body sounds as a person being monitored goes about his or her daily life.

One problem encountered in real-time acoustic physiological monitoring is parameter estimation error caused by noise and unwanted information that infects the acoustic physiological signal. Real-time acoustic physiological monitoring is often performed using a portable (e.g. wearable) device that continually analyzes an acoustic physiological signal captured by a sound transducer positioned on the body, such as the trachea, chest or back. The captured signal includes lung sounds, heart sounds and noise from body movement and the surrounding environment. Before the captured signal can be used to accurately estimate a physiological parameter, noise and unwanted information must be removed from the signal or at least reduced to a great extent. Otherwise, the result will be erroneous estimation of physiological parameters by the device and outputting of erroneous estimates. Reliance on erroneous estimates can have serious adverse consequences on the health of the person being monitored. For example, erroneous estimates can lead the person being monitored or his or her clinician to improperly interpret physiological state and cause the person to undergo treatment that is not medically indicated, or forego treatment that is medically indicated.

One physiological parameter that can be estimated in real-time acoustic physiological monitoring and plays an important role in respiratory abnormality diagnosis is inspiration-to-expiration ratio, which can be expressed as a fraction Ti/Te or a ratio I:E. Respiration in humans is typically characterized by two phases: inspiration, or the intake of air into the lungs, and expiration, or the expelling of air from the lungs. Ti/Te is computed by dividing the inspiration time by the expiration time over one breathing cycle, and is often averaged over several breathing cycles. Ti/Te can be instructive about the respiratory health of a person being monitored. For example, a healthy adult has a Ti/Te of about 0.50. For an adult suffering from severe asthma, however, Ti/Te may drop well below 0.50 due to a prolonged expiration phase caused by obstruction of the airways. Therefore, an accurate Ti/Te reading can be used as a reference to determine whether mechanical ventilatory support is needed. On the other hand, an erroneous Ti/Te reading, if relied upon, could cause a person being monitored to undertake ventilatory support when not needed or forego such support when needed.

Unfortunately, known techniques for removing or reducing non-respiratory background sounds (e.g. heart sounds, noise) from an acoustic physiological signal have either failed to isolate respiratory sounds to the extent necessary to reliably extract inspiration and expiration times, required intense computation, been unduly complex, or suffered from more than one of these shortcomings. Thus, while inspiration-to-expiration ratio extraction by real-time physiological monitoring devices from captured acoustic physiological signals can offer substantial advantages over more conventional inspiration-to-expiration ratio estimation systems (e.g. airflow detectors, rib cage movement sensors, chest expansion sensors, lung volume detectors, etc.) in terms of personal comfort, convenience and mobility, the full promise of inspiration-to-expiration ratio computation by real-time physiological monitoring devices has yet to be realized.

### SUMMARY OF THE INVENTION

The present invention provides a method and system for reliably extracting inspiration-to-expiration ratio from an acoustic physiological signal. A background sound level is set to an energy level whereat a predetermined share of data points on an energy envelope is below the energy level, after which respiration phase start and end times are determined at energy crossings above the background sound level, enabling more reliable determination of respiration phases. Moreover, reliably determined respiration phase start and end times, in addition to being used to compute inspiration-to-expiration ratio (Ti/Te), are applied to other purposes, such as computing respiration period, validating an independently computed respiration period and/or adjusting a sampling window of the acoustic physiological signal, reducing system complexity and conserving computational resources.

In one aspect of the invention, a physiological monitoring system comprises an acoustic physiological signal capture system; an acoustic physiological signal processing system communicatively coupled with the capture system; and an output interface, wherein the processing system extracts an energy envelope from an acoustic physiological signal captured by the capture system, sets a background sound level to an energy level whereat a predetermined share of data points on the energy envelope is below the energy level, identifies respiration phase start and end times based at least in part on crossings of the energy envelope above the background sound level and computes an inspiration-to-expiration ratio using the respiration phase start and end times, wherein the inspiration-to-expiration ratio is outputted on the output interface.

In some embodiments, the processing system applies a band-pass filter to the acoustic physiological signal before extracting the energy envelope.

In some embodiments, the processing system extracts the energy envelope using a standard deviation method.

In some embodiments, the processing system assigns data points on the energy envelope to different ones of a plurality of bins each spanning a discrete energy range before setting the background sound level.

In some embodiments, the processing system applies a low-pass filter to the energy envelope before setting the background sound level.

In some embodiments, the processing system applies an additional filter to the energy envelope after setting the background sound level.

In some embodiments, the processing system identifies peaks in the energy envelope after setting the background sound level.

In some embodiments, the processing system eliminates insignificant peaks in the energy envelope after identifying the peaks.

In some embodiments, the processing system computes a respiration period using the respiration phase start and end times.

In some embodiments, the processing system independently computes a respiration period and uses the respiration phase start and end times to validate the respiration period.

In some embodiments, the processing system adjusts a sampling window length of the acoustic respiratory signal using the respiration phase start and end times.

In some embodiments, the inspiration-to-expiration ratio is displayed on a user interface.

In another aspect of the invention, a physiological monitoring method comprises the steps of capturing by a physiological monitoring system an acoustic physiological signal; extracting by the system an energy envelope of the acoustic physiological signal; setting by the system a background sound level to an energy level whereat a predetermined share of data points on the energy envelope is below the energy level; identifying by the system respiration phase start and end times based at least in part on crossings of the energy envelope above the background sound level; computing by the system an inspiration-to-expiration ratio using the respiration phase start and end times; and outputting by the system the inspiration-to-expiration ratio.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a physiological monitoring system in some embodiments of the invention.

FIG. 2 shows a physiological monitoring method in some embodiments of the invention.

FIG. 3 is a plot of a raw acoustic physiological signal window.

FIG. 4 is a plot of the window of FIG. 3 after application of a band-pass filter.

FIG. 5 is a plot of an energy envelope extracted from the window of FIG. 4.

FIG. 6 is a plot of the energy envelope of FIG. 5 illustrating background sound level setting using a data binning technique.

FIG. 7 is a plot of the energy envelope of FIG. 5 illustrating inspiration and expiration start and end time identification at crossings of the energy envelope above the background sound level.

### DETAILED DESCRIPTION OF A PREFERRED EMBODIMENT

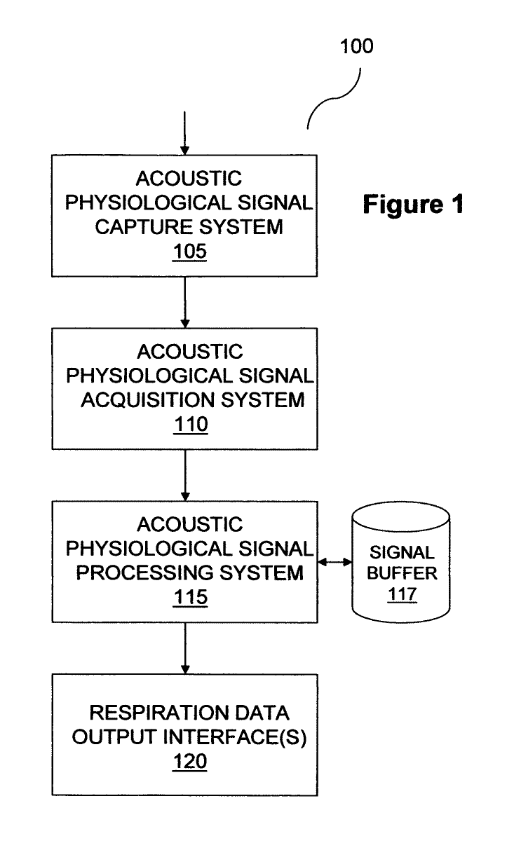

FIG. 1 shows a physiological monitoring system 100 in some embodiments of the invention. Monitoring system 100 includes an acoustic physiological signal capture system 105, an acoustic physiological signal acquisition system 110, an acoustic physiological signal processing system 115 and acoustic physiological signal output interfaces 120, communicatively coupled in series. Processing system 115 is also communicatively coupled with a signal buffer 117.

Capture system 105 detects body sounds, such as heart and lung sounds, at a detection point, such as a trachea, chest or back of a person being monitored and continually transmits an acoustic physiological signal to acquisition system 110 in the form of an electrical signal generated from detected body sounds. Capture system 105 may include, for example, a sound transducer positioned on the body of a human subject.

Acquisition system 110 amplifies, filters, performs analog/digital (A/D) conversion and automatic gain control (AGC) on the acoustic physiological signal received from capture system 105, and transmits the signal to processing system 115. Amplification, filtering, ND conversion and AGC may be performed by serially arranged pre-amplifier, band-pass filter, final amplifier, ND conversion and AGC stages, for example.

Processing system 115, under control of a processor executing software instructions, processes the acoustic physiological signal to continually estimate one or more respiration parameters of the subject being monitored. Monitored respiration parameters include inspiration-to-expiration ratio (Ti/Te or I:E) and may also include respiration period and respiration rate. To enable continual estimation of respiration parameters, processing system 115 continually buffers in signal buffer 117 and evaluates samples of the acoustic physiological signal, wherein each sample has a current sampling window length that is dynamically adjustable. Processing system 115 under control of the processor transmits to output interfaces 120 format and content information for displaying or otherwise processing information regarding recent estimates of the monitored respiration parameters. In other embodiments, processing system 115 may perform in custom logic one or more of the processing functions described herein.

Output interfaces 120 includes a user interface having a display screen for displaying information in accordance with format and content information received from processing system 115 regarding recent estimates of respiration parameters. Output interfaces 120 may also include a data management interface to an internal or external data management system that stores the information and/or a network interface that transmits the information to a remote monitoring device, such as a monitoring device at a clinician facility.

In some embodiments, capture system 105, acquisition system 110, processing system 115 and output interfaces 120 are part of a portable ambulatory monitoring device that monitors a person's physiological well-being in real-time as the person performs daily activities. In other embodiments, capture system 105, acquisition system 110, processing system 115 and output interfaces 120 may be part of separate devices that are remotely coupled via wired or wireless links.

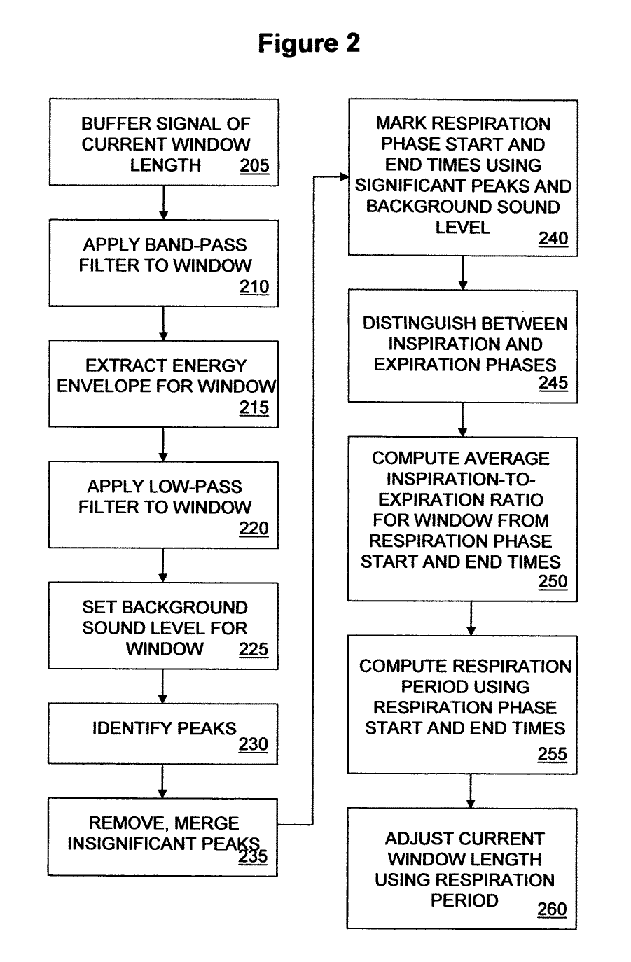

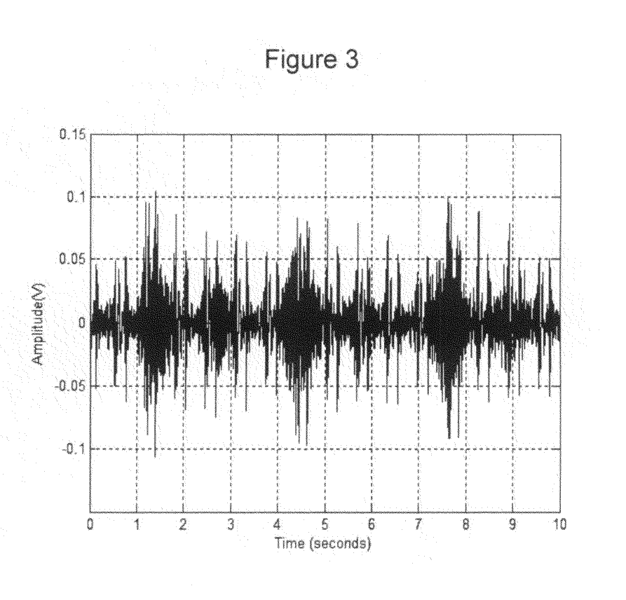

A physiological monitoring method performed by processing system 115 under processor control will now be described by reference to the flow diagram of FIG. 2 taken in conjunction with the plots of FIGS. 3-7. In the illustrated example, a primary goal of physiological monitoring is to provide real-time estimates of Ti/Te based on body sounds detected at the trachea. However, it bears noting that the method can be applied to achieve other respiratory monitoring goals, such as estimation of respiration period, validation of an independently computed respiration period, estimation of respiration rate and/or adjustment of an acoustic physiological signal sampling window length, and can achieve such goals based on detection of body sounds elsewhere on the body, such as the chest or back.

In Step 205, a window of the acoustic physiological signal of the current sample window length is stored in signal buffer 117. As the sampling window length is dynamically adjustable, signal buffer 117 is configured to be large enough to accommodate samples having a maximum sampling window length that may exceed the current sampling window length. In FIG. 3, plot of a raw acoustic physiological signal window stored in signal buffer 117 is shown. In the raw signal, lung sounds are intermingled with heart sounds and noise and are not easily distinguished.

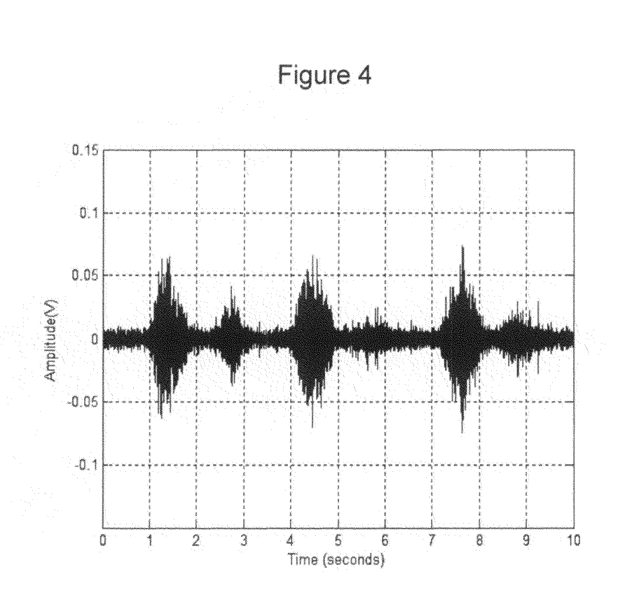

In Step 210, a band-pass filter is applied to the window to better isolate lung sounds by reducing heart sounds and noise. As lung sounds are typically found within the 300 Hz to 800 Hz frequency range, the band-pass filter may be a fifth order Butterworth filter having a high-pass cutoff frequency at 300 Hz and a low-pass cutoff frequency at 800 Hz. In FIG. 4, a plot of an acoustic physiological signal window after application of a band-pass filter is shown. Heart sounds and other noise continue to be expressed, although they are meaningfully reduced.

In Step 215, an energy envelope is extracted from the window to further improve signal-to-noise ratio. In some embodiments, a standard deviation method is used to extract the energy envelope. In an exemplary standard deviation method, the standard deviation of every N consecutive samples, which is representative of the total energy of those N samples, is computed and used as envelope data.

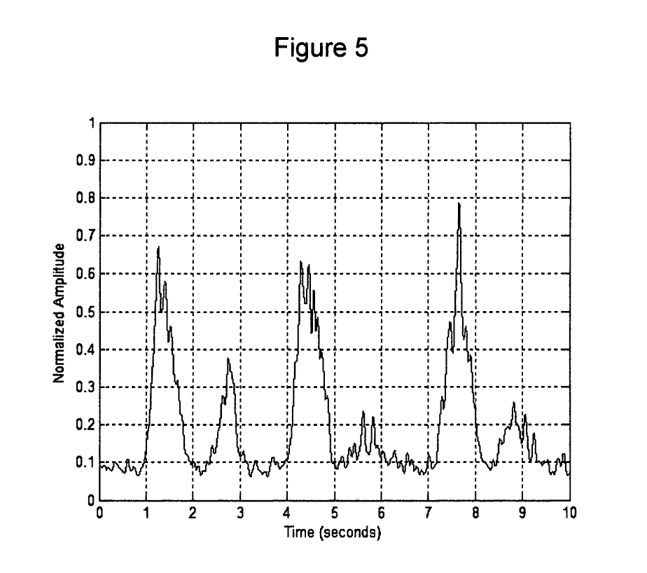

In Step 220, a low-pass filter is applied to the energy envelope to even better isolate lung sounds by further reducing heart sounds and noise. In FIG. 5, an energy envelope extracted from an acoustic physiological signal window is shown. Periodic lung sounds are clearly expressed in the energy envelope.

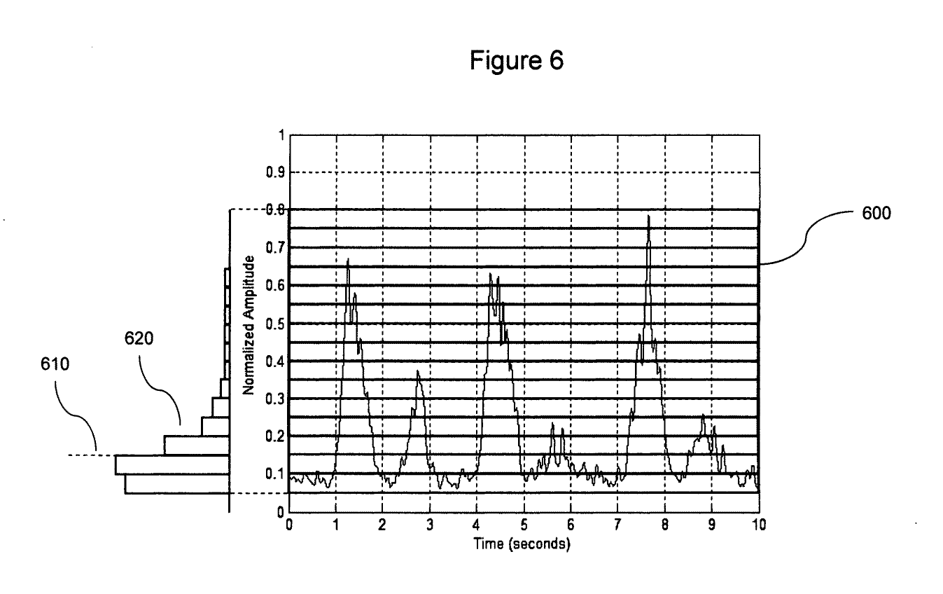

In Step 225, a background sound level of the energy envelope is set. that Background sounds tend to cluster at relatively low signal energies for relatively long signal periods. Setting a background sound level endeavors to prevent these sounds from being misidentified as respiration phase start and endpoints (i.e. start of inspiration, end of inspiration/start of expiration, end of expiration). Referring to FIG. 6, data bins 600 are first introduced to facilitate creation of an alternative description of signal energy. Each one of bins 600 spans a discrete signal energy range within the energy envelope. In the figure, fifteen bins 600 are shown covering, in the aggregate, the range from about 0.05 to 0.80 on the normalized amplitude scale; however, in practice a number of bins will be used that is sufficiently large that every data point fits within a bin. Each energy data point is assigned to the one of bins 600 within whose range the energy data point falls, and data point tallies 620 are compiled for each bin. Using tallies 620, a background sound level 610 is set to an energy level whereat a predetermined share of data points in bins 600 is below the energy level. The predetermined share may be, for example, 70%. Data points below background sound level 610 are precluded from being identified as respiration phase start and endpoints, as will be explained hereinafter.

In some embodiments, an additional filter is applied to the energy envelope at this juncture to even better isolate lung sounds by further removing short, non-respiratory energy bursts. The additional filter follows Step 225 so as not to alter the energy envelope in a manner that skews determination of background sound level 610.

In Step 230, peaks in the energy envelope that may be respiration phase peaks are identified. Data maxima at energies above background sound level 610 are identified as centers of peaks that are potential respiration phase peaks.

In Step 235, insignificant peaks among the peaks identified in Step 230 are removed or merged. Peaks that do not have at least a predetermined minimum width are disregarded as unfiltered background noise and peaks that are too close together are merged. With regard to merger, a respiration phase peak may contain gaps or dips that cause the peak to be misidentified as two or more independent peaks. Accordingly, peaks that are not separated by at least a predetermined minimum width are merged.

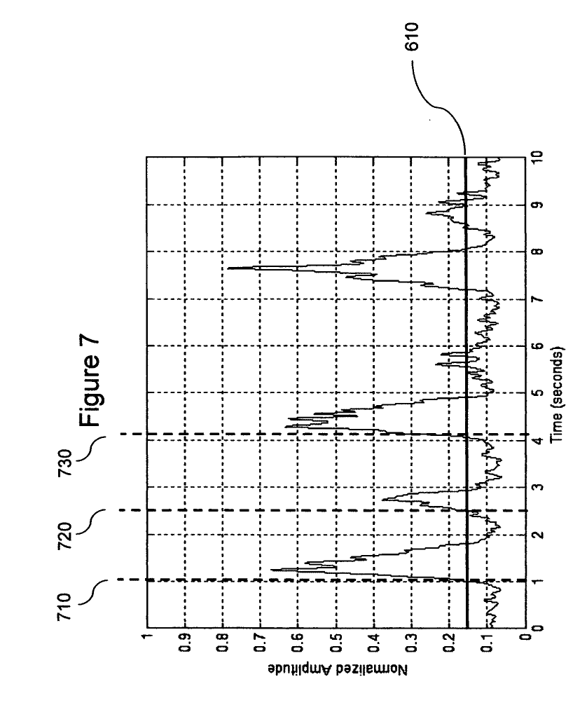

In Step 240, respiration phase start and end times are identified using significant peaks and background signal level 610. Points where the energy envelope crosses above background signal level 610 are identified as respiration phase start and end times. Turning to FIG. 7, crossing points 710, 720 and 730 are identified as respiration phase start and end times. Rising cross-points for subsequent peaks that begin at approximately five and seven seconds similarly identified as respiration phase start and end times.

At this juncture, the inspiration and expiration phases in the energy envelope have not been distinguished. Thus, it is not known which of the identified respiration phase start and end times marks the beginning of inspiration/end of expiration, and which of the identified respiration start and end times marks the end of inspiration/beginning of expiration.

In Step 245, the inspiration and expiration phases are distinguished. The user-assisted classification methods and systems described in U.S. application Ser. No. 12/386,072 entitled “Method and System for Respiratory Phase Classification Using Explicit Labeling with Label Verification,” which is incorporated herein by reference, may be invoked, by way of example.

In Step 250, Ti/Te is computed for the window from the respiration phase start and end times. Returning to FIG. 7, assume that in Step 245 respiration phase start time 710 is identified as the inspiration phase start time for a breath cycle, respiration phase start time 720 is identified as the inspiration phase end time/expiration phase start time for the breath cycle, and respiration phase start time 730 is identified as the expiration phase end time for the breath cycle. Under those assumptions, inspiration time (Ti) for the breath cycle is computed as the time difference between inspiration phase end time 720 and inspiration phase start time 710, which is approximately 1.35 seconds. Expiration time (Te) for the breath cycle is computed as the time difference between expiration phase end time 730 and expiration phase start time 720, which is approximately 1.70 seconds. In/Te for the breath cycle is therefore roughly 1.35/1.70, or 0.79. Similar calculations are made for other individual breath cycles in the window, after which an average of the individual Ti/Te values is computed.

In Step 255, the respiration period is computed using the respiration phase start and end times. The values of Ti and Te computed for individual breath cycles in Step 250 are summed to a compute respiration periods for the individual breath cycles, from which an average respiration period for the window is computed.

In Step 260, the current sampling window length is adjusted using the respiration period. Human respiration patterns vary from person-to-person and over time for the same person. The sampling window length should be long enough to capture at least one complete breath cycle for the person being monitored, but preferably capture no more than three complete breath cycles. The average respiration period computed in Step 255 is applied to adjust the current window length to meet these criteria. For example, the current window length may be selected to be twice the average respiration period.

In some embodiments, the respiration period computed in Step 255 is used to validate a respiration period computed independently by another system component. Moreover, in these embodiments the independently computed respiration period can be used to tune the maximum length of energy bursts removed by the additional filter applied after Step 225.

In some embodiments, the respiration period computed in Step 255 is used to calculate other respiration parameters, such as respiration rate in breaths per minute.

At this juncture, the flow returns to Step 205, whereat a window of the acoustic physiological signal of the new current sample window length is stored in signal buffer 117 for processing.

Processing system 115, under processor control, outputs one or more of the respiration parameters computed in the method of FIG. 2 (e.g. Ti/Te, respiration period, respiration rate) on one or more of output interfaces 120, which may include a user interface, a local analysis module, data management element and/or a network interface. For example, the Ti/Te estimate may be transmitted to a user interface whereon the estimate is displayed to the person being monitored, transmitted to a local analysis module whereon the estimate is subjected to higher level clinical processing, transmitted to a data management element whereon the estimate is logged, and/or transmitted to a network interface for further transmission to a remote analysis module or remote clinician display.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A physiological monitoring system, comprising:
an acoustic physiological signal capture system;
an acoustic physiological signal processing system communicatively coupled with the capture system; and
an output interface, wherein the processing system extracts an energy envelope from an acoustic physiological signal captured by the capture system, sets a background sound level to an energy level whereat a predetermined share of data points on the energy envelope is below the energy level, identifies respiration phase start and end times based at least in part on crossings of the energy envelope above the background sound level and computes an inspiration-to-expiration ratio using the respiration phase start and end times, wherein the inspiration-to-expiration ratio is outputted on the output interface.

2. The monitoring system of claim 1, wherein the processing system applies a band-pass filter to the acoustic physiological signal before extracting the energy envelope.

3. The monitoring system of claim 1, wherein the processing system extracts the energy envelope using a standard deviation method.

4. The monitoring system of claim 1, wherein the processing system assigns data points on the energy envelope to different ones of a plurality of bins each spanning a discrete energy range before setting the background sound level.

5. The monitoring system of claim 1, wherein the processing system applies a low-pass filter to the energy envelope before setting the background sound level.

6. The monitoring system of claim 1, wherein the processing system applies an additional filter to the energy envelope after setting the background sound level.

7. The monitoring system of claim 1, wherein the processing system identifies peaks in the energy envelope after setting the background sound level.

8. The monitoring system of claim 7, wherein the processing system eliminates insignificant peaks in the energy envelope after identifying the peaks.

9. The monitoring system of claim 1, wherein the processing system computes a respiration period using the respiration phase start and end times.

10. The monitoring system of claim 1, wherein the processing system independently computes a respiration period and uses the respiration phase start and end times to validate the respiration period.

11. The monitoring system of claim 1, wherein the processing system adjusts a sampling window length of the acoustic respiratory signal using the respiration phase start and end times.

12. The monitoring system of claim 1, wherein the inspiration-to-expiration ratio is displayed on a user interface.

13. A physiological monitoring method, comprising the steps of:
capturing by a physiological monitoring system an acoustic physiological signal;
extracting by the system an energy envelope of the acoustic physiological signal;
setting by the system a background sound level to an energy level whereat a predetermined share of data points on the energy envelope is below the energy level;
identifying by the system respiration phase start and end times based at least in part on crossings of the energy envelope above the background sound level;
computing by the system an inspiration-to-expiration ratio using the respiration phase start and end times; and
outputting by the system the inspiration-to-expiration ratio.

14. The method of claim 13, wherein the system extracts the energy envelope using a standard deviation method.

15. The method of claim 13, wherein the system assigns data points on the energy envelope to different ones of a plurality of bins each spanning a discrete energy range before setting the background sound level.

16. The method of claim 13, wherein the system computes a respiration period using the respiration phase start and end times.

17. The method of claim 13, wherein the system independently computes a respiration period and uses the respiration phase start and end times to validate the respiration period.

18. The method of claim 13, wherein the system adjusts a sampling window length of the acoustic respiratory signal using the respiration phase start and end times.

19. The method of claim 13, wherein the system displays the inspiration-to-expiration ratio.
